(19) United States
(12) Patent Application Publication
(10) Pub. No.: US 2026/0018273 A1
(43) Pub. Date: Jan. 15, 2026

(54) INFORMATION PROCESSING METHOD AND INFORMATION PROCESSING SYSTEM

(71) Applicant: Panasonic Intellectual Property Management Co., Ltd., Osaka (JP)

(72) Inventor: TERUHISA OKUYA, Osaka (JP)

(21) Appl. No.: 19/333,418

(22) Filed: Sep. 19, 2025

Related U.S. Application Data
(63) Continuation of application No. PCT/JP2024/008992, filed on Mar. 8, 2024.

(30) Foreign Application Priority Data
Apr. 4, 2023 (JP) 2023-060957

(51) Int. Cl. G16H 20/70; A61B 5/16

(52) U.S. Cl. CPC G16H 20/70; A61B 5/165

(57) ABSTRACT

An information processing system executes, on a first user, a first environmental control previously performed on users with attributes similar to attributes of the first user; determines whether a psychological score of the first user has increased as a result of the first environmental control; and determines, if it is determined that the psychological score has not increased, a second environmental control different from the first environmental control as the next environmental control to be executed on the first user. The second environmental control is an environmental control that was previously performed on users with attributes similar to attributes of the first user and can contribute to an increased psychological score.

### BACKGROUND

### 1. Technical Field

The present disclosure relates to a technique for controlling the environment of a space where a user is present.

### 2. Description of the Related Art

Japanese Unexamined Patent Application Publication No. 2021-16137 discloses a technique which involves calculating an index from the user's state or the state of user's surroundings, inputting the index into a device control algorithm constituted by a neural network to determine a control content for a device, and optionally setting the device control algorithm for each of user attributes, such as age, gender, and occupation.

### SUMMARY

Japanese Unexamined Patent Application Publication No. 2021-16137 merely discloses operating the device in accordance with the control content determined by the device control algorithm and evaluating the determined control content, and does not disclose changing the control content in accordance with the result of the evaluation. To efficiently determine the control content more suitable for each individual user, the technique disclosed in Japanese Unexamined Patent Application Publication No. 2021-16137 requires further improvement.

One non-limiting and exemplary embodiment provides a technique for efficiently determining an environmental control more suitable for each individual user.

In one general aspect, the techniques disclosed here feature an information processing method in a computer. The information processing method includes deriving a psychological score indicating a degree of a psychological state of a first user; executing, on the first user, a first environmental control previously performed on users with attributes similar to attributes of the first user; determining whether the psychological score of the first user has increased as a result of the first environmental control; and determining, if it is determined that the psychological score has not increased, a second environmental control different from the first environmental control as the next environmental control to be executed on the first user. The second environmental control is an environmental control that was previously performed on users with attributes similar to attributes of the first user and can contribute to an increased psychological score.

According to the present disclosure, an environmental control more suitable for each individual user can be determined efficiently.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram illustrating a configuration of an information processing system according to an embodiment of the present disclosure;

FIG. 2 is a diagram illustrating a data configuration of a personal database;

FIG. 3 is a diagram illustrating a data configuration of a group database;

FIG. 4 is a flowchart illustrating a process of the information processing system illustrated in FIG. 1;

FIG. 5 is an explanatory diagram illustrating a first exemplary determining method for determining whether an increase condition is satisfied;

FIG. 6 is an explanatory diagram illustrating a second exemplary determining method for determining whether an increase condition is satisfied;

FIG. 7 is another explanatory diagram illustrating the second exemplary determining method for determining whether an increase condition is satisfied;

FIG. 8 is an explanatory diagram illustrating a process of determining an optimal threshold;

FIG. 9 is an explanatory diagram illustrating another process of determining an optimal threshold;

FIG. 10 is a diagram illustrating an example of a display screen presenting an intervention effect;

FIG. 11 is a diagram illustrating an example of another display screen presenting an intervention effect;

FIG. 12 is a diagram illustrating another example of the display screen presenting an intervention effect;

FIG. 13 is a diagram illustrating an example of a display screen presenting an intervention effect in relation to a threshold;

FIG. 14 is a diagram illustrating another example of the display screen presenting an intervention effect in relation to a threshold; and

FIG. 15 is a diagram illustrating an example of a selection screen.

### DETAILED DESCRIPTIONS

### (Underlying Knowledge Forming Basis of One Aspect of the Present Disclosure)

Researches have been conducted on applying appropriate stimuli to users at work by controlling the environment of the space where the users are present, so as to enhance the users' concentration or creativity and thereby improve their work productivity.

Tasks in the workplace can be broadly classified into two types: concentration tasks, which have a single answer, and creative tasks, which generate multiple answers or ideas.

In concentration tasks, it is particularly important to continuously sustain concentration over time, but the duration of concentration varies from one individual to another. Similarly, in creative tasks, it is important to continuously sustain the state of exhibiting creativity over time, but this duration also varies from one individual to another. In the present specification, the term concentration may be defined as an index indicating the degree to which convergent thinking is exhibited. The state of exhibiting creativity may be defined as a state in which divergent thinking is being exhibited. The state of exhibiting creativity may be defined as a state in which at least one of originality, fluency, or flexibility is being exhibited.

As described above, the duration of concentration or creativity varies from one user to another. Moreover, a stimulus that improves concentration or creativity also varies from one user to another. Therefore, to sustain concentration or creativity for a long time, it is necessary to optimize both the type of stimulus and the timing of its delivery for each individual user. When environmental control is performed on the basis of user information, it is difficult to optimize with 100% accuracy because changes occur depending on the mood or state of the user. When the optimal type of stimulus and timing of its delivery for each individual user are determined on the basis of the intervention history of stimuli to the user, it is not possible to apply the optimal stimulus from the initial use. Moreover, a large amount of intervention history needs to be accumulated. Therefore, this technique lacks efficiency.

The technique disclosed in Japanese Unexamined Patent Application Publication No. 2021-16137 involves evaluating the control content determined by the device control algorithm. However, this evaluation is made for the purpose of adjusting the weights of intermediate layers of the neural network that constitutes the machine control algorithm, and not for the purpose of determining whether to apply a different stimulus. Therefore, to determine a control content more suitable for each individual user, the technique disclosed in Japanese Unexamined Patent Application Publication No. 2021-16137 requires further improvement.

The present inventor has found that, by determining an environmental control suitable for a first user through a trial-and-error process on the basis of an environmental control applied to other users with attributes similar to those of the first user and the psychological score of the first user, an environmental control more suitable for each individual user can be efficiently determined. On the basis of this finding, the present inventor has conceived various aspects of the present disclosure.

Hereinafter, aspects of the present disclosure will be described.
(1) An information processing method in a computer according to an aspect of the present disclosure includes deriving a psychological score indicating a degree of a psychological state of a first user; executing, on the first user, a first environmental control previously performed on users with attributes similar to attributes of the first user; determining whether the psychological score of the first user has increased as a result of the first environmental control; and determining, if it is determined that the psychological score has not increased, a second environmental control different from the first environmental control as the next environmental control to be executed on the first user. The second environmental control is an environmental control that was previously performed on users with attributes similar to attributes of the first user and can contribute to an increased psychological score.

With this configuration, if the psychological score of the first user has not increased by executing the first environmental control, the second environmental control different from the first environmental control is executed. Since the environmental control suitable for the first user is thus determined by trial and error, an environmental control that is more suitable for each individual user can be determined. Moreover, since both the first environmental control and the second environmental control are environmental controls for users with attributes similar to those of the first user, it is possible to efficiently determine the content of environmental control without accumulating a large amount of historical data for the first user.
(2) In the information processing method according to (1), the determining the environmental control may include determining, if it is determined that the psychological score has increased, a content of the next environmental control to be executed on the first user on the basis of a content of the first environmental control.

With this configuration, if the psychological score of the first user has increased as a result of executing the first environmental control, the content of the next environmental control to be executed on the first user is determined on the basis of the content of the first environmental control. Therefore, an environmental control more suitable for the first user can be determined more efficiently.
(3) In the information processing method according to (1) or (2), the first environmental control and the second environmental control each may include a control that applies, to the first user, a stimulus related to at least one sense selected from the group consisting of senses of vision, hearing, smell, and touch, and may include a control that changes a content of the stimulus in response to a decrease in the psychological score of the first user.

With this configuration, if the psychological score of the first user has decreased, a stimulus related to at least one sense selected from the group consisting of senses of vision, hearing, smell, and touch is changed. A stimulus that can contribute to an increased psychological score of the first user can thus be determined.
(4) In the information processing method according to (3), the control that changes the content of the stimulus in response to a decrease in the psychological score of the first user may include a control that changes the content of the stimulus when the psychological score of the first user falls below a threshold determined in association with a content of the first environmental control.

With this configuration, the stimulus is changed when the psychological score of the first user falls below the threshold.
(5) In the information processing method according to (4), the determining the environmental control may include determining, if the psychological score has increased, an environmental control with a different threshold as a content of the next environmental control to be executed on the first user, without changing the stimulus applied by the first environmental control.

With this configuration, when the first environmental control can contribute to an increased psychological score of the first user, it is possible to adjust the intervention frequency of the stimulus while maintaining the content of the stimulus applied by the first environmental control. A threshold that achieves an appropriate intervention frequency can thus be determined.
(6) In the information processing method according to any one of (3) to (5), the determining the environmental control may include determining, if it is determined that the psychological score has increased, an environmental control that applies to a user a stimulus similar to the stimulus applied by the first environmental control as a content of the next environmental control to be executed on the first user.

With this configuration, when the first environmental control can contribute to an increased psychological score of the first user, a stimulus similar to the stimulus applied by the first environmental control is applied to the first user. This makes it possible to search for a better stimulus in the first environmental control.
(7) In the information processing method according to any one of (1) to (6), the determining whether the psychological score of the first user has increased as a result of the first environmental control may include comparing the amount of past increase in the psychological score of users with attributes similar to attributes of the first user, resulting from the first environmental control previously performed, with the amount of increase in the psychological score of the first user, resulting from the first environmental control; and determining that the psychological score of the first user has increased as a result of the first environmental control when the amount of increase in the psychological score of the first user exceeds or is substantially the same as the amount of past increase in the psychological score.

With this configuration, a determination of whether the psychological score of the first user has increased can be made by taking into account the relation between the amount of increase in the psychological score of the first user and the amount of increase in the psychological score of users with attributes similar to those of the first user.
(8) In the information processing method according to any one of (1) to (7), the second environmental control determined when it is determined that the psychological score has not increased may be an environmental control that yields the second largest increase in the psychological score after the first environmental control, among environmental controls previously performed on users with attributes similar to attributes of the first user.

With this configuration, an environmental control that is highly likely to increase the psychological score of the first user can be efficiently determined.
(9) In the information processing method according to any one of (1) to (8), the psychological score may indicate a degree of user's concentration or creativity.

With this configuration, the concentration or creativity of the first user can be improved.
(10) The information processing method according to any one of (1) to (9) may further include recording contents of environmental controls executed on individual users in a personal database in association with the attributes of the first user.

This configuration enables construction of a personal database that records historical data of environmental controls executed on users.
(11) The information processing method according to (10) may further include generating a group database by organizing the contents of environmental controls recorded in the personal database in accordance with the attributes of the users. The group database indicates the contents of environmental controls corresponding to the attributes.

With this configuration, a group database that records the contents of environmental controls corresponding to attributes is generated. This makes it possible to quickly determine the environmental control that can contribute to an increased psychological score of the first user.
(12) The information processing method according to any one of (1) to (11) may further include presenting, to the first user, a display screen indicating the psychological score derived when the first environmental control or the second environmental control is executed.

This configuration allows the first user to recognize the reason for executing the first environmental control or the second environmental control, and gives the first user a sense of understanding.
(13) An information processing system according to another aspect of the present disclosure includes a processor. The processor executes a process involving deriving a psychological score indicating a degree of a psychological state of a first user; executing, on the first user, a first environmental control previously performed on users with attributes similar to attributes of the first user; determining whether the psychological score of the first user has increased as a result of the first environmental control; and determining, if it is determined that the psychological score has not increased, a second environmental control different from the first environmental control as the next environmental control to be executed on the first user. The second environmental control is an environmental control that was previously performed on users with attributes similar to attributes of the first user and can contribute to an increased psychological score.

This configuration provides an information processing system that can efficiently determine an environmental control that is more suitable for each individual user.

Such an information processing program may be distributed via a computer-readable non-transitory storage medium, such as a CD-ROM, or via a communication network, such as the Internet.

Embodiments described below each illustrate an example of the present disclosure. Numerical values, shapes, components, steps, the order of the steps, and the like that are described in the following embodiments are merely examples, and not intended to limit the present disclosure. Of the components described in the following embodiments, those not mentioned in the independent claims, which represent the highest-level concepts, are described as optional components. The contents of any of the embodiments may be combined.

### EMBODIMENTS

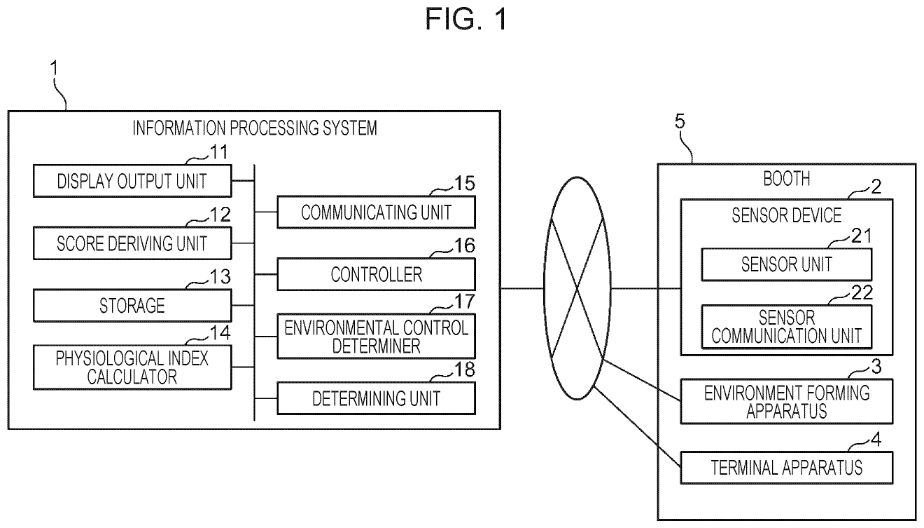

FIG. 1 is a block diagram illustrating a configuration of an information processing system 1 according to an embodiment of the present disclosure. The information processing system 1 is communicatively connected via a network to a sensor device 2, an environment forming apparatus 3, and a terminal apparatus 4. The information processing system 1 is constituted by a computer, such as a cloud server or an edge server. When the information processing system 1 is a cloud server, the network is constituted, for example, by the Internet, whereas when the information processing system 1 is an edge server, the network is constituted, for example, by a local area network.

The sensor device 2, the environment forming apparatus 3, and the terminal apparatus 4 are installed in a booth 5. The booth 5 is a space where users work. The users work using computers and the like in the booth 5. As described above, tasks in the workplace can be broadly classified into two types: concentration tasks and creative tasks.

The sensor device 2 is a device that acquires user's biometric data. The sensor device 2 includes a sensor unit 21 and a sensor communication unit 22. The sensor unit 21 is, for example, a cerebral blood flow sensor, a camera, an electrocardiogram sensor, or an electroencephalogram sensor. The cerebral blood flow sensor is constituted, for example, by a near-infrared sensor (NIRS) and detects hemoglobin concentration. The camera captures the user's face or posture. The electrocardiogram sensor includes electrodes and detects the user's heart rate and the like. The electroencephalogram sensor includes, for example, electrodes and detects the user's brain waves

The sensor communication unit 22 is constituted, for example, by a communication circuit and transmits biometric data detected by the sensor unit 21 to the information processing system 1.

The environment forming apparatus 3 is an apparatus that stimulates the user by forming an environment in the booth 5. The environment forming apparatus 3 is communicatively connected via a network to the information processing system. The environment forming apparatus 3 includes a lighting device, a speaker, an air conditioner, a scent diffuser, and a display device. The lighting device is, for example, a ceiling light or a desk lamp and adjusts the color and intensity of illuminating light in the booth 5. The speaker outputs sound. The air conditioner regulates the temperature and humidity in the booth 5. The scent diffuser is a device that releases various scents. The scents released include coffee, peach, forest, mint, and other scents. The display device displays images to form a visual environment in the booth 5. The lighting device and the display device stimulate the user's sense of vision, the speaker stimulates the user's sense of hearing, the air conditioner stimulates the user's sense of touch, and the scent diffuser stimulates the user's sense of smell.

The terminal apparatus 4 is constituted, for example, by a portable computer or a desktop computer and is an apparatus that the user possesses. The terminal apparatus 4 may be an apparatus that the user uses for work. The terminal apparatus 4 includes, for example, an operating unit, a display, a processor, and a communication circuit and is communicatively connected via the network to the information processing system 1.

The information processing system 1 includes a display output unit 11, a score deriving unit 12, a storage 13, a physiological index calculator 14, a communicating unit 15, a controller 16, an environmental control determiner 17, and a determining unit 18.

The display output unit 11 generates a display screen to be presented to the user.

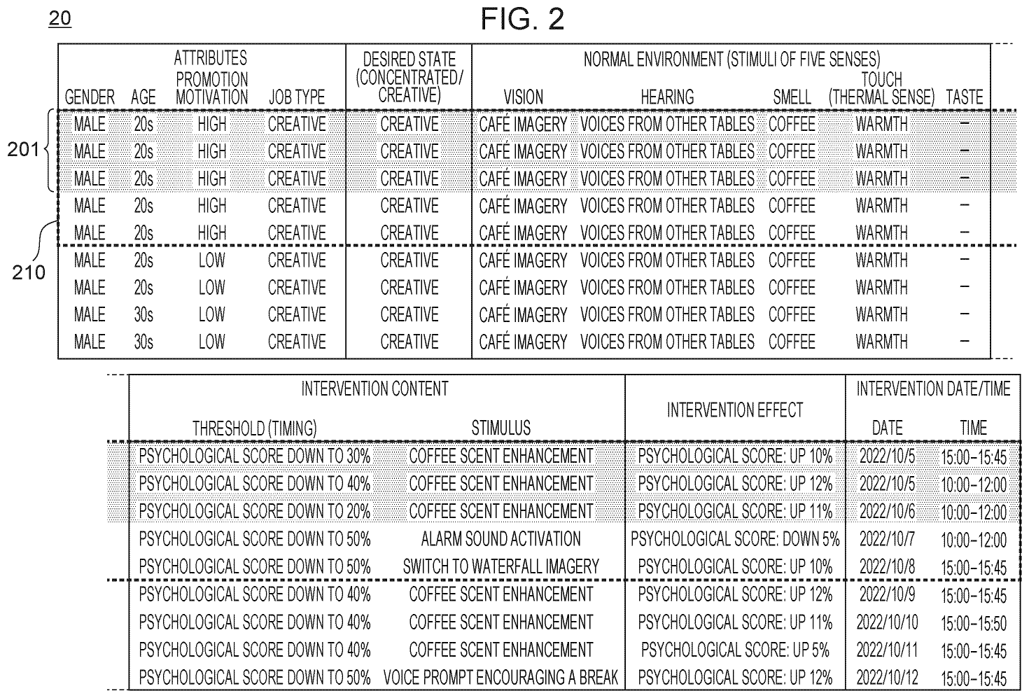

The score deriving unit 12 derives a psychological score indicating the user's state on the basis of a physiological index calculated by the physiological index calculator 14. The psychological score includes a psychological score of concentration and a psychological score of creativity. The psychological score of concentration is a psychological score that indicates the level of concentration of the user. The psychological score of creativity is a psychological score that indicates the degree of creativity exhibited by the user. The score deriving unit 12 derives a psychological score of concentration for users whose desired state is “concentrated”, and derives a psychological score of creativity for users whose desired state is “creative”. The desired state is stored in advance in a personal database 20 illustrated in FIG. 2. The psychological score may be expressed as a continuous numerical value ranging from, for example, 1 to 100. The psychological score may be expressed in binary form. An example of binary representation of the psychological score is 1 for creativity and 0 for no creativity, and 1 for concentration and 0 for no concentration. The psychological score may be expressed as “creative” and “not creative”, and “concentrated” and “not concentrated”.

As techniques for calculating the degree of concentration, those described in Japanese Unexamined Patent Application Publication No. 2020-8278 (Document D1), International Publication No. 2012/150657 (Document D2), and Japanese Unexamined Patent Application Publication No. 2020-201755 (Document D3) are known. The score deriving unit 12 may derive, as a psychological score of concentration, the degree of concentration calculated by using any of the techniques described in Documents D1 to D3.

For example, Document D1 discloses a technique in which an image of a user captured by a camera is input into an image-processing neural network to estimate the user's state, including the user's movement, blink frequency, gender, and age, and then the estimated user's state is input into a concentration-estimation neural network to calculate the degree of concentration. Therefore, when the technique disclosed in Document D1 is adopted, the score deriving unit 12 may derive the psychological score of concentration by using, as a physiological index, the user's state estimated in an image processing network.

Document D2 discloses a technique in which hemoglobin concentration indicating the amount of cerebral blood flow is calculated from biometric data measured by a near-infrared sensor, and then the degree of concentration is calculated on the basis of the hemoglobin concentration. Therefore, when the technique disclosed in Document D2 is adopted, the score deriving unit 12 may derive the psychological score of concentration by using the hemoglobin concentration as a physiological index.

Document D3 discloses a technique in which a habitual posture of a user during concentration is detected from an image of the user captured by a camera, and the degree of concentration is calculated on the basis of the detected habitual posture. Therefore, when the technique disclosed in Document D3 is adopted, the score deriving unit 12 may derive the user's psychological score of concentration by using, as a physiological index, data indicating the habitual posture during concentration.

As techniques for calculating a degree to which creativity is exhibited, those described in Japanese Unexamined Patent Application Publication No. 2020-16943 (Document D4) and “Cerebral blood flow associated with creative performance: A comparative study” NeuroImage 38 (2007) 519-528 (Document D5) are known. The score deriving unit 12 may derive a psychological score of creativity using the technique described in either Document D4 or D5.

Document D4 discloses a technique in which a power spectrum of time-series data of heartbeat intervals is calculated, a degree of change in balance between sympathetic and parasympathetic nervous system activities is derived from the power spectrum, and a degree to which creativity is exhibited is calculated from the degree of change. Therefore, when the technique disclosed in Document D4 is adopted, the score deriving unit 12 may derive the psychological score of creativity by using a heart rate index (described below) as a physiological index.

Document D5 discloses a technique in which a degree to which creativity is exhibited is calculated from the amount of cerebral blood flow. Therefore, when the technique disclosed in Document D5 is adopted, the score deriving unit 12 may derive the psychological score of creativity by using the amount of cerebral blood flow as a physiological index.

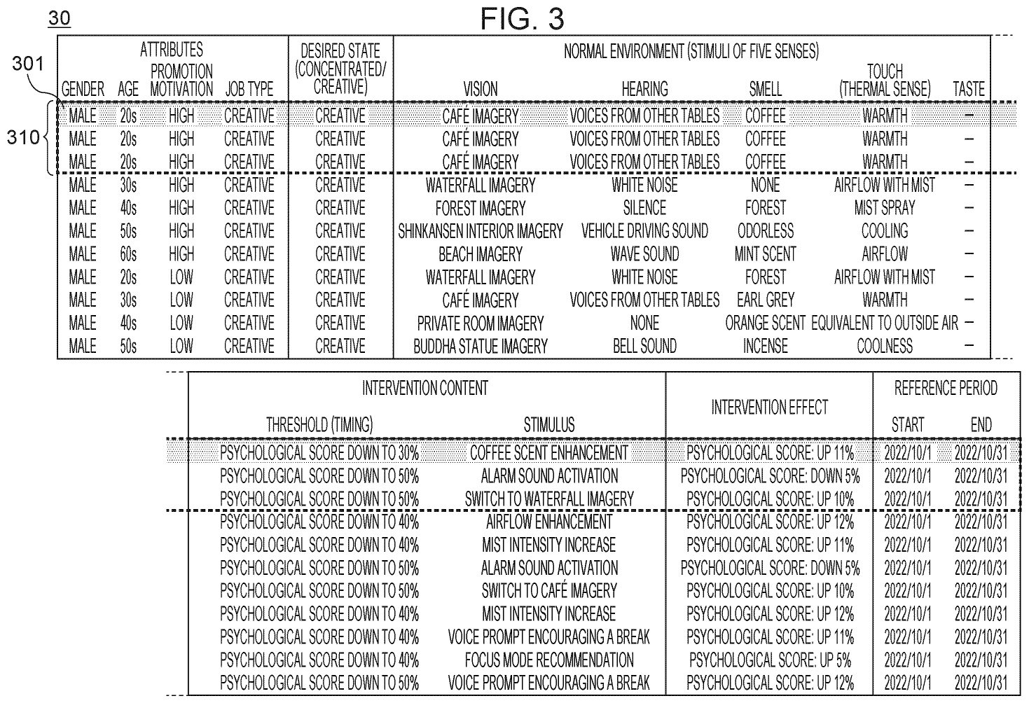

The storage 13 is constituted by a non-volatile rewritable storage device, such as a hard disk drive or a solid-state drive. The storage 13 stores the personal database 20 illustrated in FIG. 2 and a group database 30 illustrated in FIG. 3.

The physiological index calculator 14 calculates a physiological index on the basis of biometric data transmitted by the sensor device 2. The physiological index is an index used when the score deriving unit 12 derives a psychological score. Examples of the physiological index include the user's state described in Document D1, the hemoglobin concentration described in Document D2, the data indicating the habitual posture described in Document D3, the heart rate index described in Document D4, and the amount of cerebral blood flow described in Document D5. Examples of the heart rate index include intervals between R-waves (RRI) and the coefficient of variation of R-R intervals (CvRR).

The communicating unit 15 is a communication circuit that connects the information processing system 1 to the network. The communicating unit 15 receives biometric data from the sensor device 2. The communicating unit 15 transmits a control signal to the environment forming apparatus 3. The communicating unit 15 transmits display data for a display screen to the terminal apparatus 4. The communicating unit 15 receives a result of user's selection from the terminal apparatus 4.

The controller 16 is responsible for the overall control of the information processing system 1.

The environmental control determiner 17 executes, on a first user, a first environmental control previously performed on users with attributes similar to those of the first user. The first user is a user present in the booth 5. Attributes refer to information indicating user characteristics, such as gender, age, promotion motivation, and job type. Promotion motivation is an indicator of the level of user's enthusiasm for work. Promotion motivation may be expressed in stages, such as “high” and “low”. Job type refers to the type of user's occupation, such as creative work or simple work. Specifically, the environmental control determiner 17 may identify environmental controls for users with attributes similar to those of the first user from the group database 30, and determine a first environmental control from among the identified environmental control. The environmental control determiner 17 records the content of the environmental control executed on the first user in the personal database 20, in association with the attributes of the first user.

If the determining unit 18 determines that the psychological score of the first user has not increased as a result of execution of the first environmental control on the first user, the environmental control determiner 17 determines a second environmental control different from the first environmental control as the next environmental control to be executed on the first user. The second environmental control is an environmental control that was previously performed on users with attributes similar to those of the first user and can contribute to an increased psychological score. Specifically, the environmental control determiner 17 may identify environmental controls for users with attributes similar to those of the first user from the group database 30, and determine the second environmental control from the identified environmental controls. The environmental control determiner 17 transmits a control signal for executing the determined first or second environmental control to the environment forming apparatus 3 using the communicating unit 15.

The determining unit 18 determines whether the psychological score derived by the score deriving unit 12 has increased by executing the first environmental control on the first user.

In FIG. 1, the display output unit 11, the score deriving unit 12, the physiological index calculator 14, the controller 16, the environmental control determiner 17, and the determining unit 18 may be implemented by execution of an information processing program by a processor, or may be implemented by dedicated hardware circuits.

FIG. 2 is a diagram illustrating a data configuration of the personal database 20. The personal database 20 records historical data of environmental controls executed on each individual user. One record in the personal database 20 is generated each time an intervention (described below) is executed, and corresponds to one piece of historical data. The personal database 20 includes items, such as attributes, desired state, normal environment (stimuli of the five senses), intervention content, intervention effect, and date and time of use.

The item “attributes” contains each user's gender, age, promotion motivation, and job type. The attributes are entered by the user in advance. The desired state is a state that the user actually wants to be in or is estimated to want to be in. The desired state may be determined, for example, from the content of work indicated by the user's schedule registered in a scheduler, or may be directly entered by the user. For example, if the content of work is to take meeting minutes, the desired state is determined to be “concentrated”. For example, if the content of work is to generate ideas, the desired state is determined to be “creative”. The desired state may be determined from the user's current brain wave state. For example, if alpha waves are present for a certain period or longer, the desired state is determined to be “creative”. The period during which alpha waves are present is calculated, for example, by analyzing the brain waves detected by the electroencephalogram sensor. These determinations of the desired state are executed, for example, by the controller 16. The controller 16 records the results of the determinations in the personal database 20.

“Normal environment” indicates the environmental control applied to the user in a normal state. “Normal environment” includes stimuli of the five senses: vision, hearing, smell, touch (thermal sense), and taste. The record in the first row contains vision “café imagery”, hearing “voices from other tables”, smell “coffee”, and touch “warmth”. This record indicates that a café imagery was displayed on the display device, a sound simulating conversation from other tables was output from the speaker, a coffee scent was released from the scent diffuser, and an airflow at a predetermined warm temperature was output from the air conditioner. The normal state is a state in which the psychological score does not drop to or below the threshold.

“Intervention content” is the content of a stimulus applied to the user when the psychological score drops to or below the threshold. Applying this stimulus to the user is referred to as an intervention. “Intervention content” includes a threshold and a stimulus. The threshold is the value of a threshold at which the intervention was executed. The record in the first row contains “psychological score down to 30%” as the threshold. This indicates that the intervention was executed when the psychological score dropped to 30% of its maximum value. The maximum value of the psychological score refers to the maximum value of the psychological score that has ever been derived for each individual user. The stimulus refers to the content of the stimulus applied to the user at the time of the intervention. The record in the first row contains “coffee scent enhancement”. This indicates that an intervention to enhance the coffee scent was performed.

“Intervention effect” indicates the effect of the intervention. The record in the first row contains “psychological score: up 10%”. This indicates that the psychological score after the intervention (which may hereinafter be referred to as the post-intervention psychological score) increased by 10% relative to the psychological score before the intervention (which may hereinafter be referred to as the pre-intervention psychological score). The post-intervention psychological score relative to the pre-intervention psychological score is expressed as ((“post-intervention psychological score”−“pre-intervention psychological score”)/“pre-intervention psychological score”)×100.

“Intervention date/time” indicates when the intervention was executed. For example, the record in the first row contains “2022 Oct. 5 15:00-15:45”. This indicates that the intervention was executed in the period from 15:00 to 15:45 on Oct. 5, 2022. This makes it possible to optimize stimuli by taking into account effective types of stimuli that depend on the time of year (e.g., trend or season-dependent stimuli).

In the example illustrated in FIG. 2, the desired state of all users is “creative”, so the psychological score represents the psychological score of creativity. When the desired state is “concentrated”, the psychological score of concentration is calculated as the psychological score.

The controller 16 generates the group database 30 that indicates the contents of environmental controls corresponding to attributes, by organizing the contents of environmental controls recorded in the personal database 20 in accordance with attributes.

FIG. 3 is a diagram illustrating a data configuration of the group database 30. The group database 30 contains the same items as the personal database 20, except that the group database 30 contains “reference period” instead of “intervention date/time”, and therefore the explanation is omitted. “Reference period” includes the start and end dates and times of the period referred to in determining the environmental control for the first user.

The controller 16 generates the group database 30 in the following manner. Referring to FIG. 2, a record group 210 is a collection of records of users with the same attributes. The record group 210 consists of records of the users whose attributes are: gender “male”, age “20s”, promotion motivation “high”, and job type “creative”. The controller 16 extracts the record group 210 from the personal database 20. The controller 16 classifies the records in the extracted record group 210 by intervention content “stimulus”. A record set 201 consisting of the first to third rows shares the same intervention content “stimulus”. Therefore, the controller 16 consolidates the record set 201 into a single record 301 (see FIG. 3). The record 301 records “psychological score down to 30%” as the intervention content “threshold”. This is because “30%” was calculated as the representative value of the intervention content “threshold” of “30%”, “40%”, and “20%” in the record set 201. The representative value is either the mean or the median. The record 301 records “psychological score: up 11%” as the intervention effect. This is because “11%” was calculated as the representative value of the intervention effect of “10%”, “12%”, and “11%” in the record set 201. The controller 16 executes this process on the other record groups to generate the group database 30. A record group 310 is a collection of record sets from the record group 210. The record group 310 contains records of users that are the same in attributes but differ in intervention content “stimulus”.

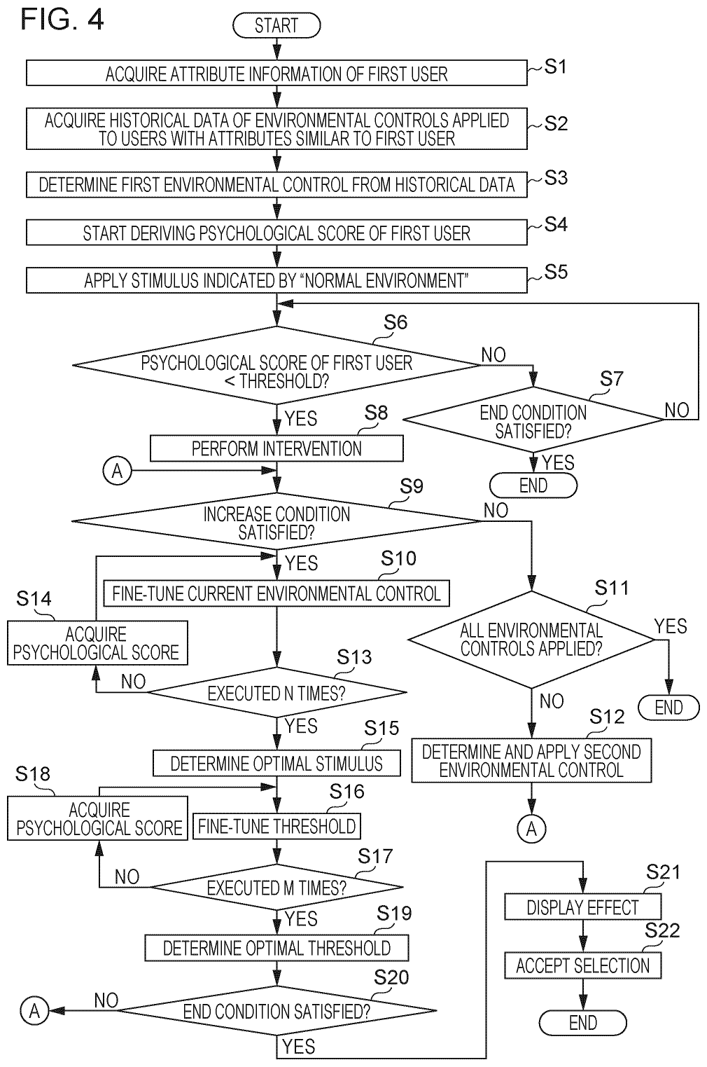

FIG. 4 is a flowchart illustrating a process of the information processing system 1 illustrated in FIG. 1. The controller 16 acquires attribute information indicating the attributes of the first user (step S1). The controller 16 may acquire the attribute information of the first user by extracting, from the personal database 20, the attribute information corresponding to a user ID transmitted from the terminal apparatus 4. In this case, each record in the personal database is assumed to be associated with the user ID (not illustrated). The terminal apparatus 4 may acquire the user ID by prompting the user to enter the user ID.

Next, the environmental control determiner 17 acquires, from the group database 30, historical data of environmental controls previously performed on users with attributes similar to those of the first user (step S2). Referring to FIG. 3, when the attributes of the first user are gender “male”, age “20s”, promotion motivation “high”, and job type “creative”, the attributes of three pieces of historical data indicated by the record group 310 in the first to third rows match the attributes of the first user, so the environmental control determiner 17 acquires these three pieces of historical data. If attributes that exactly match the attributes of the first user are not stored in the group database 30, the environmental control determiner 17 may identify, among multiple record groups 310, the record group 310 most similar to the attributes of the first user, and extract historical data contained in the identified record group 310. In this case, the environmental control determiner 17 may determine that the similarity is higher when a greater number of attribute items (including gender, age, promotion motivation, and job type) match between each of the multiple record groups 310 and the first user.

Next, the environmental control determiner 17 determines, from the historical data acquired in step S2, the historical data with the highest intervention effect as the first environmental control (step S3). In the example illustrated in FIG. 3, the historical data in the first row has the highest intervention effect among the historical data in the first to third rows, and therefore the historical data in the first row is determined as the first environmental control.

Next, the score deriving unit 12 starts deriving the psychological score of the first user (step S4). In this case, the score deriving unit 12 derives the psychological score using the physiological index calculated using any of the techniques described above. In this example, the psychological score of creativity is derived, as the desired state of the first user is “creative”.

Next, in the first environmental control determined in step S3, the environmental control determiner 17 applies a stimulus indicated by “normal environment” to the first user (step S5). In the example illustrated in FIG. 3, the historical data in the first row contains vision “café imagery”, hearing “voices from other tables”, smell “coffee”, and touch “warmth” as “normal environment”, and these four stimuli are applied to the first user. Specifically, the environmental control determiner 17 applies these four stimuli to the first user by transmitting a control signal for each of “café imagery”, “voices from other tables”, “coffee scent”, and “warmth” to the environment forming apparatus 3 using the communicating unit 15. The intensity of the coffee scent applied to the first user, and the intensity (set temperature) of the air conditioner corresponding to the warmth are determined in advance. Although all the four stimuli recorded in the “normal environment” are applied in this case, one or more of these four stimuli or three or fewer of these four stimuli, randomly determined, may be applied to the first user.

Next, the environmental control determiner 17 determines whether the psychological score of the first user derived by the score deriving unit 12 is below a threshold. If the psychological score of the first user is not below the threshold (NO in step S6), the process proceeds to step S7. On the other hand, if the psychological score of the first user is below the threshold (YES in step S6), the process proceeds to step S8. The threshold adopted is one that corresponds to the first environmental control determined in step S3. Here, since the historical data in the first row illustrated in FIG. 3 is determined as the first environmental control, “psychological score down to 30%” is adopted as the threshold. Therefore, if the psychological score of the first user falls below 30%, it is determined that the psychological score of the first user has fallen below the threshold.

Next, the environmental control determiner 17 determines whether an end condition is satisfied (step S7). The end condition is, for example, that the current time reaches the scheduled end time of use of the booth 5 by the first user, or that the first user has entered an operation requesting the end of the environmental control. If the end condition is not satisfied (NO in step S7), the process returns to step S6. That is, in the case of NO in step S6 and NO in step S7, the stimulus applied in step S5 remains effective and continues to be applied. If the end condition is satisfied (YES in step S7), the process ends.

Next, the environmental control determiner 17 performs an intervention (step S8). In this case, since “coffee scent enhancement” is recorded as the intervention content “stimulus” in the historical data in the first row illustrated in FIG. 3, an intervention to enhance the coffee scent is performed. A predetermined value is used to determine the degree to which the intensity of the stimulus (which may hereinafter be referred to as the stimulus intensity) is to be increased.

Next, the environmental control determiner 17 determines whether the psychological score of the first user satisfies an increase condition (step S9). Details of this determination will be described below.

If the psychological score of the first user does not satisfy the increase condition (NO in step S9), the environmental control determiner 17 determines whether all environmental controls previously applied to users with attributes similar to those of the first user have been applied (step S11). If all the environmental controls have been applied to the first user (YES in step S11), the process ends because there were no environmental controls that allow the psychological score to satisfy the increase condition. For example, if the environmental controls in the record group 310 are applied to the first user in descending order of intervention effects and the psychological score still does no satisfy the increase condition even after application of the environmental control with the lowest intervention effect to the first user, then the determination is YES in step S11. If the process ends in this case, a message prompting the first user to use another booth 5 may be presented.

If not all the environmental controls have been applied (NO in step S11), the environmental control determiner 17 determines a second environmental control and applies the determined second environmental control to the first user (step S12). In this case, the historical data with the next highest intervention effect among the historical data indicated by the record group 310 acquired in step S2 is determined as the second environmental control. Here, the historical data in the first row illustrated in FIG. 3 is determined as the first environmental control, and since the historical data in the third row has the next highest intervention effect in the record group 310, the historical data in the third row is determined as the second environmental control. In the example illustrated in FIG. 3, the historical data in the third row contains vision “café imagery”, hearing “voices from other tables”, smell “coffee”, and touch “warmth” as “normal environment”, and these four stimuli are applied to the first user. Since the intervention content is recorded as being that the coffee scent is enhanced when the psychological score drops to 20%, an intervention to enhance the coffee scent is performed when the psychological score drops to 20%. As in the case of the first environmental control, the environmental control is applied by transmitting various control signals from the communicating unit 15 to the environment forming apparatus 3. After completion of step S12, the process returns to step S9.

If the psychological score of the first user satisfies the increase condition in step S9 (YES in step S9), the environmental control determiner 17 fine-tunes the current environmental control (step S10). This fine-tunes the first or second environmental control that is effective for the first user. The fine-tuned environmental control is an example of environmental control that provides the user with a stimulus similar to that provided by the first environmental control or the second environmental control.

Next, the environmental control determiner 17 determines whether the fine-tuning of the environmental control has been performed N times (where N is an integer) (step S13). If the fine-tuning has not been performed N times (NO in step S13), the environmental control determiner 17 acquires the psychological score derived by the score deriving unit 12 (step S14) and returns the process to step S10. If the fine-tuning has been performed N times (YES in step S13), the process proceeds to step S15.

Next, the environmental control determiner 17 determines the optimal stimulus for the first user from the results of steps S10, S13, and S14 (step S15). Details of the operation in steps S10, S13, S14, and S15 will be described below.

Next, the environmental control determiner 17 fine-tunes the threshold (step S16). This fine-tuning is the process of searching for an optimal threshold for the optimal stimulus determined in step S15.

Next, the environmental control determiner 17 determines whether the fine-tuning of the threshold has been performed M times (where M is an integer) (step S17). If the fine-tuning has not been performed M times (NO in step S17), the environmental control determiner 17 acquires the psychological score derived by the score deriving unit 12 (step S18) and returns the process to step S16. If the fine-tuning has been performed M times (YES in step S17), the process proceeds to step S19.

Next, the environmental control determiner 17 determines the optimal threshold for the first user from the results of steps S16, S17, and S18 (step S19). Details of the operation in steps S16, S17, S18, and S19 will be described below.

Next, the environmental control determiner 17 determines whether the end condition is satisfied (step S20). The end condition will not be described in detail, as it is the same as that in step S7. If it is determined that the end condition is satisfied (YES in step S20), the process proceeds to step S21. On the other hand, if it is determined that the end condition is not satisfied (NO in step S20), the process returns to step S9.

Next, the display output unit 11 displays, on the display of the terminal apparatus 4, a display screen indicating the intervention effect (step S21). Details of this operation will be described below.

Next, the display output unit 11 acquires an operation of selecting the content of environmental control from the first user through the terminal apparatus 4 (step S22). Details of this operation will be described below.

### (Details of Operation in Step S9)

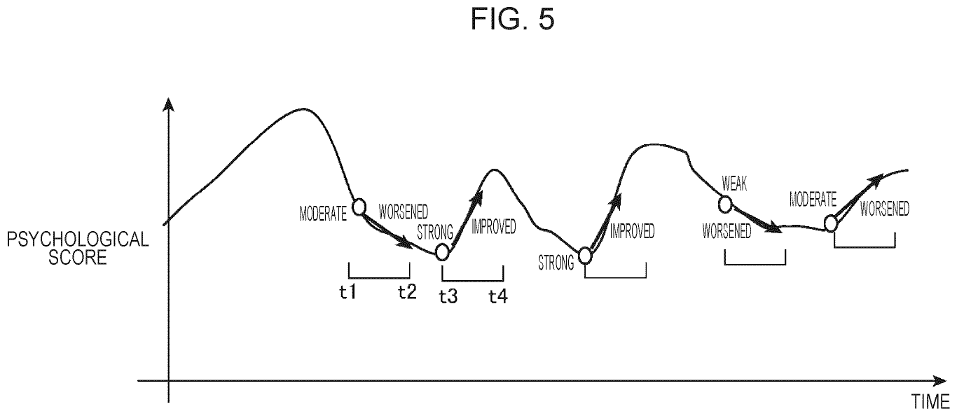

Next, details of a method for determining whether an increase condition is satisfied (which may hereinafter be simply referred to as the determining method) in step S9 of FIG. 4 will be described. There are first and second exemplary determining methods. FIG. 5 is an explanatory diagram illustrating the first exemplary determining method for determining whether an increase condition is satisfied. In FIG. 5, the vertical axis represents the psychological score, and the horizontal axis represents time. In the first exemplary determining method, the determination of whether the psychological score has increased is made on the basis of whether the rate of increase in psychological score (which may hereinafter be referred to as the psychological score increase rate) after the intervention, relative to before the intervention, exceeds a threshold. For example, at time t1, the environmental control determiner 17 performs an intervention with a stimulus intensity “moderate”. At time t2, which is after the elapse of a predetermined period from time t1, the environmental control determiner 17 calculates the psychological score increase rate. The increase rate is calculated by ((“post-intervention psychological score”−“pre-intervention psychological score”)/“pre-intervention psychological score”)×100. Since the psychological score increase rate at time t2 after the intervention is lower than the threshold and the psychological score worsens, it is determined that the increase condition is not satisfied. In this case, the determination is NO in step S9. At time t3, an intervention with the stimulus intensity “strong” is performed. At time t4, which is after the elapse of the predetermined period from time t3, the psychological score increase rate is greater than the threshold and the psychological score improves. Therefore, it is determined that the increase condition is satisfied. In this case, the determination is YES in step S9. Here, the predetermined period may be a value set by the user, or may be a value determined in advance.

As described above, in the first exemplary determining method, a determination as to whether the psychological score has increased is made on the basis of whether the psychological score increase rate exceeds the threshold. It is thus easy to determine whether the psychological score has increased.

Next, the second exemplary determining method for determining whether an increase condition is satisfied will be described. In the second exemplary determining method, the environmental control determiner 17 compares a past psychological score increase rate of users with attributes similar to those of the first user, resulting from an environmental control previously performed, with a psychological score increase rate of the first user, resulting from the environmental control. If the psychological score increase rate of the first user exceeds or is substantially the same as the past psychological score increase rate of users with attributes similar to those of the first user, the environmental control determiner 17 determines that the psychological score of the first user has increased as a result of the environmental control. Note that the psychological score increase rate is an example of the amount of increase in psychological score.

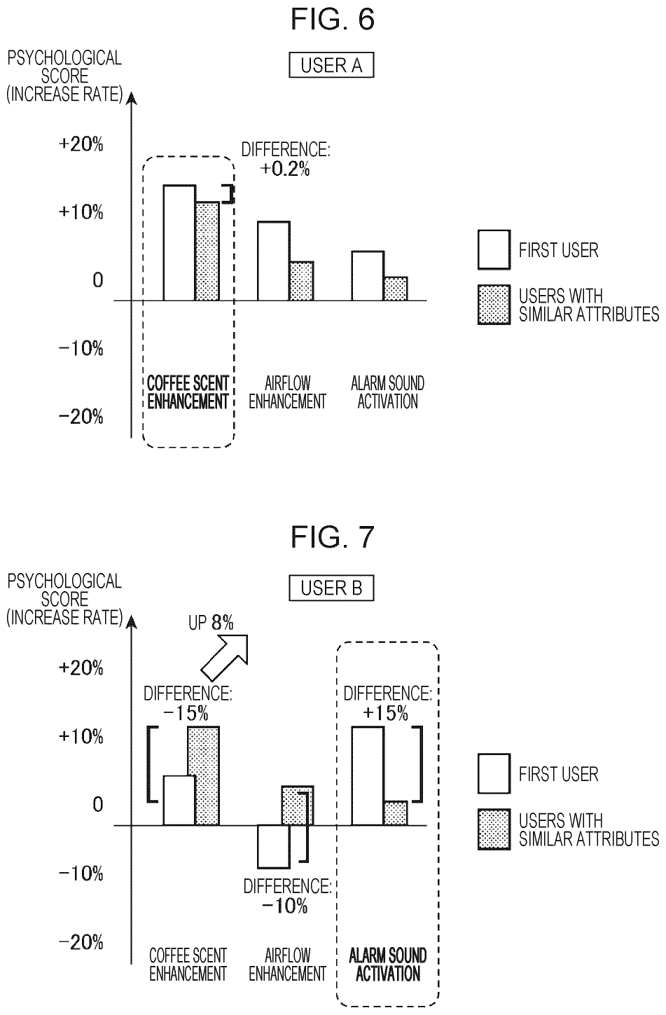

FIG. 6 and FIG. 7 are explanatory diagrams each illustrating the second exemplary determining method. In FIG. 6, the vertical axis represents the psychological score increase rate, and the horizontal axis represents the type of stimulus. In FIG. 6, a white bar represents the increase rate of the first user, and a shaded bar represents the mean or median of the increase rates of users with attributes similar to those of the first user. The same applies to the graphs of FIG. 7 to FIG. 9. The first user is User A in FIG. 6. In FIG. 6, when an intervention to enhance the intensity of the coffee scent stimulus was performed on User A, the psychological score increase rate of User A was 0.2% higher than that of users with similar attributes. In this case, the environmental control determiner 17 determines that the current environmental control satisfies the increase condition.

The first user is User B in FIG. 7. In the example illustrated in FIG. 7, when an intervention to enhance the intensity of the coffee scent stimulus was performed on User B, the psychological score increase rate was 15% lower than that of users with similar attributes although the psychological score was improved. In this case, the environmental control determiner 17 determines that the current environmental control does not satisfy the increase condition. Similarly, when an intervention to enhance the airflow was performed on User B, the psychological score worsened, and the psychological score increase rate was 10% lower than that of users with similar attributes. In this case, the environmental control determiner 17 determines that this environmental control for User B does not satisfy the increase condition. On the other hand, when an intervention to activate an alarm sound was performed on User B, the psychological score improved and the psychological score increase rate was 15% higher than that of users with similar attributes. In this case, the environmental control determiner 17 determines that the current environmental control satisfies the increase condition. Thus, with the second exemplary determining method, a determination of whether the psychological score of the first user has increased can be made by taking into account the relation between the amount of increase in the psychological score of the first user and the amount of increase in the psychological score of users with attributes similar to those of the first user.

### (Details of Operation in Steps S10, S13, S14, and S15)

The process of fine-tuning the environmental control in steps S10, S13, S14, and S15 of FIG. 4 will now be described. If the most effective environmental control for users with similar attributes continues to be applied, it is not possible to determine the type of stimulus and the corresponding threshold that will have a high intervention effect for each individual user. In the present disclosure, therefore, the type of stimulus with a high intervention effect is searched for in the environmental control currently applied to the first user. Specifically, the type of stimulus with a high intervention effect is searched for among the stimuli presented in “normal environment” in the environmental control currently applied to the first user.

First, the environmental control determiner 17 randomly extracts any one or more stimulus sets from the stimulus types presented in “normal environment” indicated by the current environmental control which serves as the base. In the example of the first row in FIG. 3, one or more stimulus sets are extracted from vision “café imagery”, hearing “voices from other tables”, smell “coffee”, and touch “warmth”.

Next, the environmental control determiner 17 applies stimuli indicated by a stimulus set to the first user, and acquires a psychological score after the elapse of a predetermined period. The environmental control determiner 17 performs such a process for each stimulus set. From the psychological score corresponding to each stimulus set, the environmental control determiner 17 determines the optimal stimulus type for the first user. For example, the environmental control determiner 17 may determine, as the optimal stimulus type, the stimulus set that yields the highest increase rate of the post-intervention psychological score, relative to the pre-intervention psychological score.

### (Details of Operation in Steps S16, S17, S18, and S19)

The operation in steps S16, S17, S18, and S19 is an operation that determines the optimal threshold for the first user for the optimal stimulus determined in step S15. First, the environmental control determiner 17 determines a threshold by increasing or decreasing the current threshold in predetermined increments within a predetermined range. Then, if the psychological score of the first user falls below the threshold determined, the environmental control determiner 17 applies the optimal stimulus determined in step S15 to the first user. Next, the environmental control determiner 17 acquires the psychological score of the first user after the elapse of a predetermined period following the application of the stimulus. The environmental control determiner 17 repeats the process of acquiring the psychological score while varying the threshold. Then, the environmental control determiner 17 determines the optimal threshold on the basis of the psychological score acquired for each threshold.

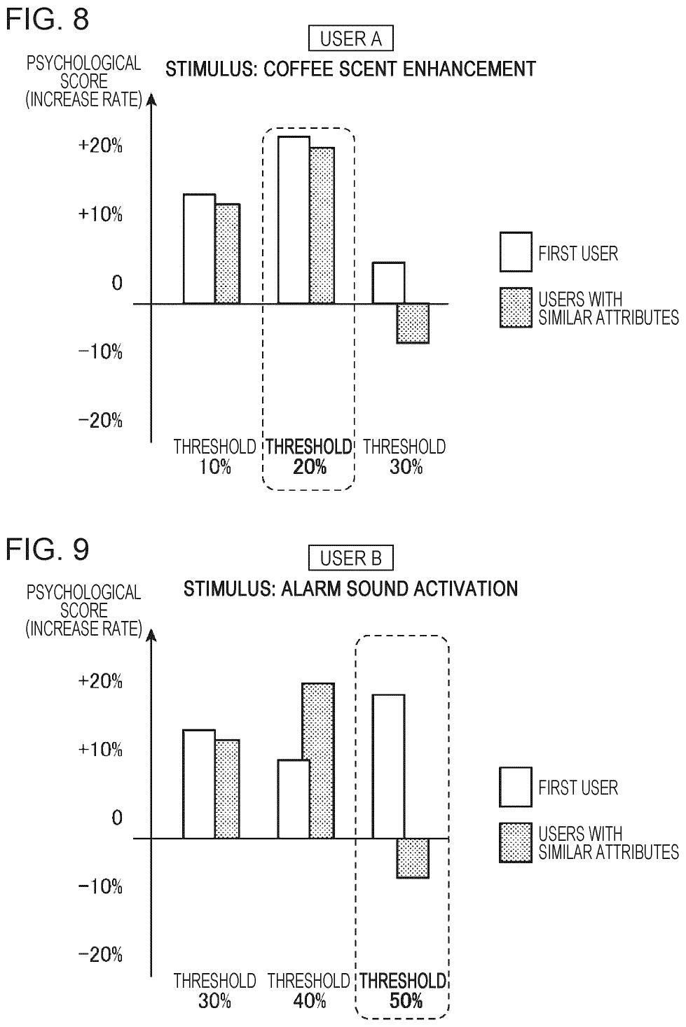

FIG. 8 and FIG. 9 are explanatory diagrams each illustrating a process of determining an optimal threshold. In the example of FIG. 8, the first user is User A and “coffee scent enhancement” is determined as the optimal stimulus for User A. The example of FIG. 8 illustrates the psychological score increase rate of User A and the psychological score increase rate of users with similar attributes when the threshold for “coffee scent enhancement” is 10%, 20%, and 30%. The threshold that yields the highest psychological score increase rate for users with similar attributes is “20%”. The psychological score increase rate of User A at the threshold “20%” is higher than the psychological score increase rate of users with similar attributes. Therefore, the threshold “20%” is determined as the optimal threshold for User A.

In the example of FIG. 9, the first user is User B and “alarm sound activation” is determined as the optimal stimulus for User B. The example of FIG. 9 illustrates the psychological score increase rate of User B and the psychological score increase rate of users with similar attributes when the threshold for “alarm sound activation” is 30%, 40%, and 50%. The threshold that yields the highest psychological score increase rate for users with similar attributes is “40%”. The psychological score increase rate of User B at the threshold “40%” is lower than the psychological score increase rate of users with similar attributes. Therefore, it is determined that the threshold “40%” is not the optimal threshold for User B. For users with similar attributes, the threshold that yields the second highest psychological score increase rate is “30%”. The psychological score increase rate of User B at this threshold is lower than the psychological score increase rate of users with similar attributes. Therefore, it is determined again that the threshold “30%” is not the optimal threshold for User B. On the other hand, the psychological score increase rate of User B at the threshold “50%” is higher than the psychological score increase rate of users with similar attributes. Therefore, the threshold “50%” is determined as the optimal threshold for User B.

As described above, in the examples of FIG. 8 and FIG. 9, the environmental control determiner 17 compares, in descending order of the psychological scores of users with similar attributes, the psychological score increase rate of users with similar attributes with the psychological score increase rate of the first user. Then, if the psychological score increase rate of the first user is higher than the psychological score increase rate of users with similar attributes, the environmental control determiner 17 determines the corresponding threshold as the optimal threshold for the first user.

Although a threshold is searched for in the examples of FIG. 8 and FIG. 9, a stimulus intensity may be searched for in addition to, or instead of, the threshold using a method similar to that used to search for the threshold. In the example of FIG. 9, the frequency of alarm sound may be searched for using a method similar to that used to search for the threshold. In the examples of FIG. 8 and FIG. 9, a threshold is searched for the optimal stimulus determined in step S15. Alternatively, the optimal threshold may be searched for the current environmental control before fine-tuning. In this case, the operation in steps S10, S13, S14, and S15 may be omitted.

### (Details of Operation in Steps S21 and S22)

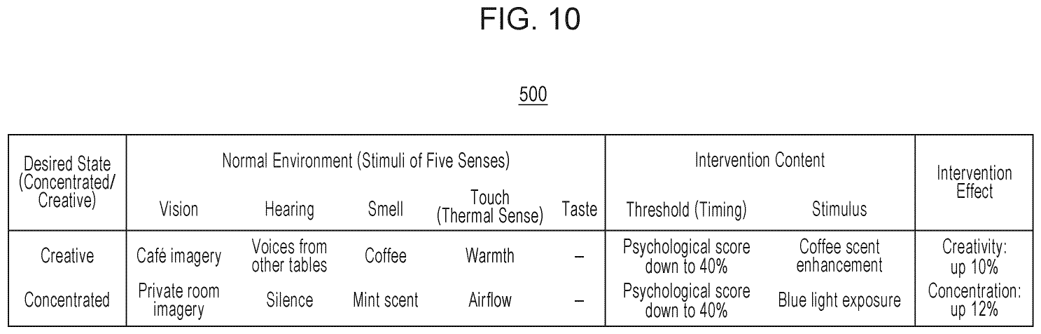

FIG. 10 is a diagram illustrating an example of a display screen 500 presenting an intervention effect. The display screen 500 displays the contents of optimal environmental controls determined for User A in a tabular form. The display screen 500 includes columns for “desired state”, “normal environment”, “intervention content”, and “intervention effect”. Details of these items have been described with reference to FIG. 2 and FIG. 3, so the description will be omitted here. In this example, the contents of the optimal environmental controls for both “creative” and “concentrated” are presented. For example, for “creative”, the display screen 500 indicates that when the psychological score drops to 40%, an intervention to enhance the coffee scent improves the psychological score by 10%. By referring to the display screen 500, User A can objectively understand the environmental control that is suitable for User A.

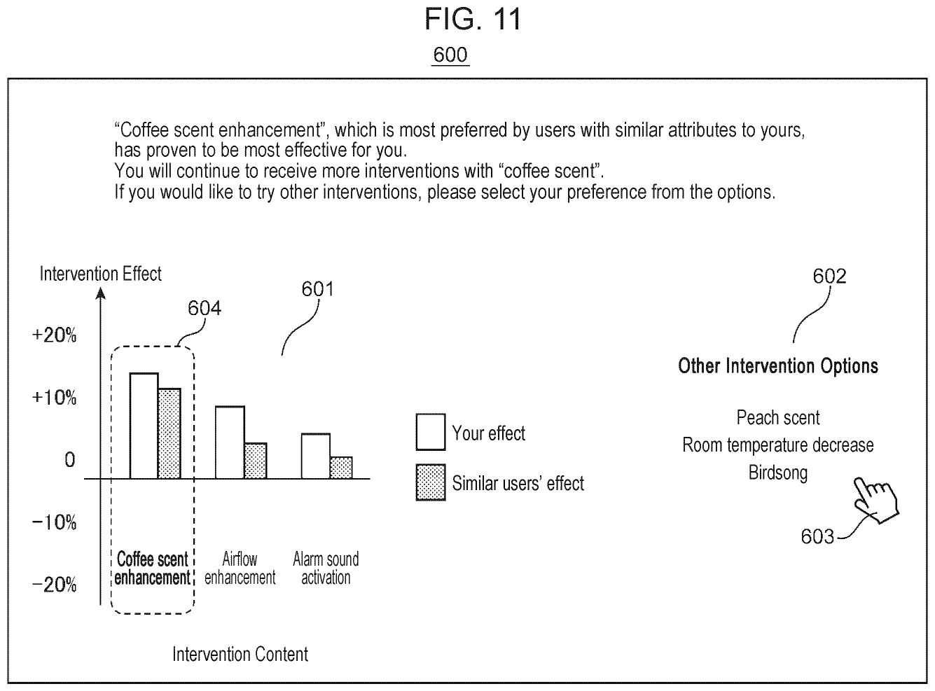

FIG. 11 is a diagram illustrating an example of a display screen 600 presenting an intervention effect. This display screen 600 is for User A. The display screen 600 includes a graph display area 601 and a selection area 602. The display screen 600 displays a message indicating that the intervention effect of “coffee scent enhancement” was the highest, that more coffee scent interventions will be applied the next time User A uses the booth 5, and that the intervention content can be changed.

The graph display area 601 displays a bar graph indicating, for each of the interventions applied to User A, the intervention effect for User A and the intervention effect for users with similar attributes. Here, the psychological score increase rate is used to evaluate the intervention effect. Since the “coffee scent enhancement” was determined as the optimal intervention in this example, bars representing the intervention effects of “coffee scent enhancement” for User A and users with similar attributes are enclosed by a dotted line 604.

The selection area 602 is an area that allows User A to select a preferred intervention from among other intervention options. For example, the selection area 602 displays the second to fourth most effective interventions for users with similar attributes. If User A wishes to select an intervention other than “coffee scent enhancement”, User A selects an intervention from those displayed in the selection area 602 by operating an icon 603. The selected intervention is stored in the storage 13 and determined as the intervention for the next time User A uses the booth 5.

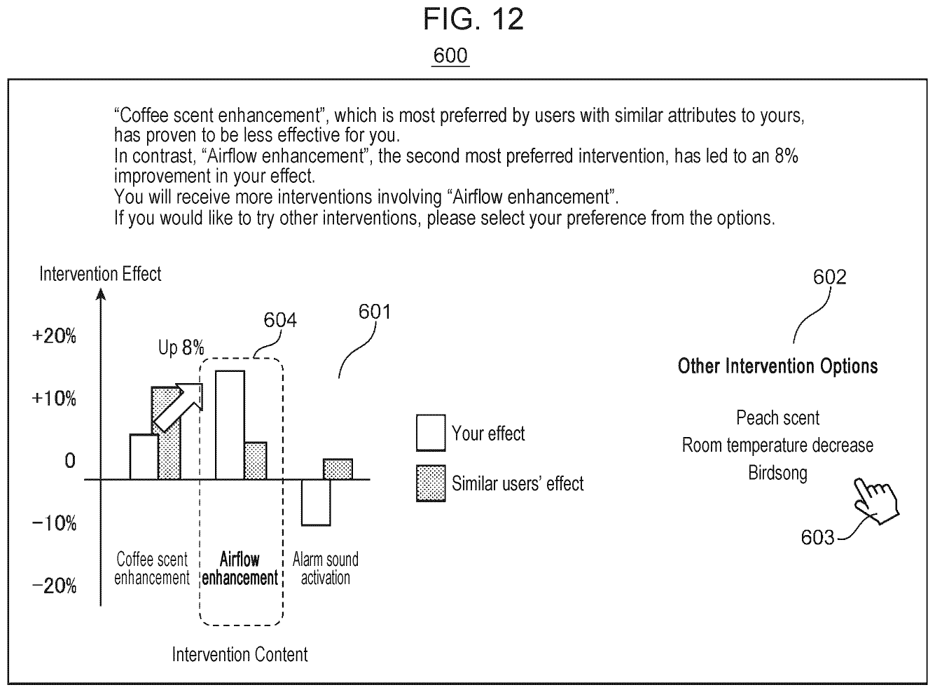

FIG. 12 is a diagram illustrating another example of the display screen 600 presenting an intervention effect. This display screen 600 is for User B. The display screen 600 displays a message indicating that the “coffee scent enhancement”, which is the most effective intervention for users with similar attributes, was less effective for User B, that the intervention effect has improved with “airflow enhancement”, that more interventions involving “airflow enhancement” will be applied the next time User B uses the booth 5, and that the intervention content can be changed.

Since “airflow enhancement” was determined as the optimal intervention in the example illustrated in FIG. 12, bars representing the effects of “airflow enhancement” for User B and users with similar attributes are enclosed by the dotted line 604. As compared to “coffee scent enhancement”, which was the most effective intervention for users with similar attributes, “airflow enhancement” increased the intervention effect for User B by 8%. An arrow and a message indicating this are displayed on the display screen 600. Other than this, the display screen 600 in FIG. 12 is the same as that in FIG. 11, and thus the description will be omitted.

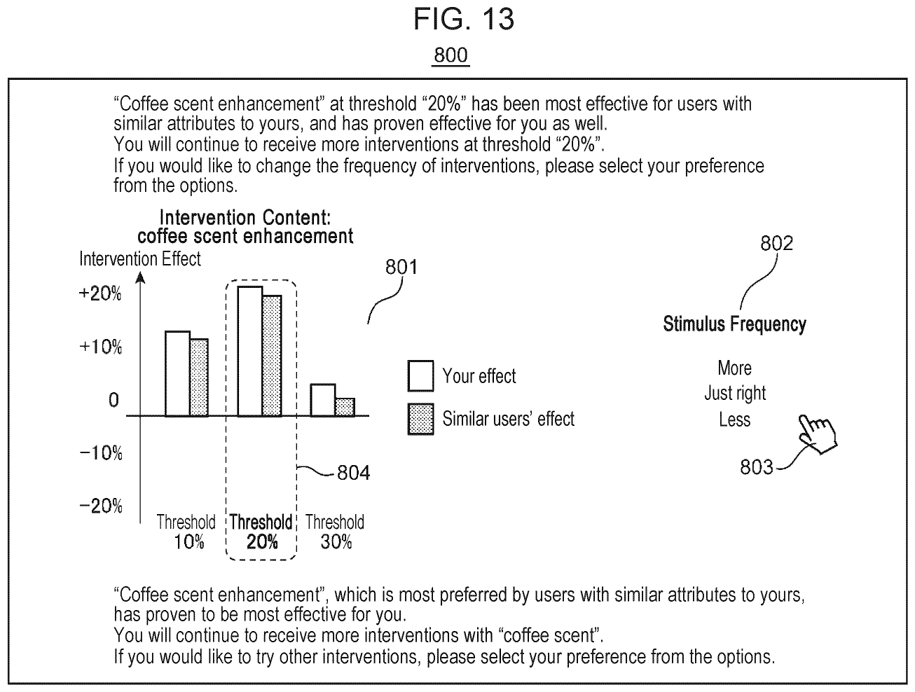

FIG. 13 is a diagram illustrating an example of a display screen 800 presenting an intervention effect in relation to a threshold. This display screen 800 is for User A. The display screen 800 displays a message indicating that the intervention effect of “coffee scent enhancement” was highest at the threshold “20%”, that more interventions at the threshold “20%” will be applied the next time User A uses the booth 5, and that the intervention frequency can be changed. The display screen 800 includes a graph display area 801 and a selection area 802.

The graph display area 801 displays a bar graph indicating, for each of the thresholds applied to User A, the intervention effect for User A and the intervention effect for users with similar attributes. Here, the psychological score increase rate is used to evaluate the intervention effect. Since the threshold “20%” was determined as the optimal threshold in this example, the bars representing the intervention effects for User A and users with similar attributes at the threshold “20%” are enclosed by a dotted line 804.

The selection area 802 is an area that allows User A to select an intervention frequency. In the selection area 802, User A can select one of “more”, “just right”, and “less” as the intervention frequency. If “more” is selected, the optimal threshold determined for User A is increased by a predetermined value. This increases the intervention frequency. If “just right” is selected, the optimal threshold determined for User A is maintained. The intervention frequency thus remains unchanged. If “less” is selected, the optimal threshold determined for User A is decreased by a predetermined value. This reduces the intervention frequency. If User A wishes to change the intervention frequency, User A selects an intervention frequency displayed in the selection area 802 by operating an icon 803. The selected intervention frequency is stored in the storage 13 and determined as the intervention frequency for the next time User A uses the booth 5. The lower area of the display screen 800 displays a message indicating that “coffee scent enhancement” has been selected as the optimal intervention.

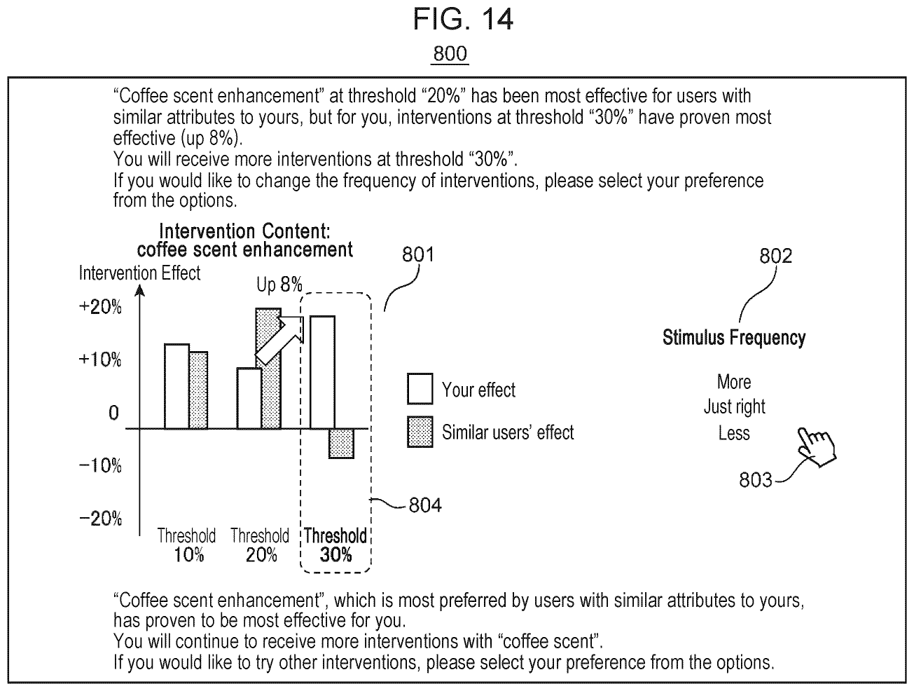

FIG. 14 is a diagram illustrating another example of the display screen 800 presenting an intervention effect in relation to a threshold. This display screen 800 is for User C (first user). The display screen 800 displays a message indicating that for “coffee scent enhancement”, the intervention effect for users with similar attributes was highest at the threshold “20%”, that the intervention effect for User C was highest at the threshold “30%”, that more interventions at the “threshold “30%” will be applied the next time User C uses the booth 5, and that the intervention frequency can be changed.

Since the threshold “30%” was determined as the optimal threshold in the example illustrated in FIG. 14, the bars representing the intervention effects for User C and users with similar attributes at the threshold “30%” are enclosed by the dotted line 804 in the graph display area 801. As compared to the threshold “20%”, which had the highest intervention effect for users with similar attributes, the threshold “30%” increased the intervention effect for User C by 8%. An arrow and a message indicating this are displayed on the display screen 800. Other than this, the display screen 800 in FIG. 14 is the same as that in FIG. 13, and thus the description will be omitted.

The display output unit 11 may display one of the display screens 500, 600, and 800, or may display all the display screens in a switchable manner.

As described above, in the information processing system according to the present embodiment, if the psychological score of the first user has not increased after execution of a first environmental control, a second environmental control different from the first environmental control is executed. Furthermore, in the first or second environmental control, the optimal intervention for the user is determined. Thus, in the present embodiment, the content of environmental control suitable for the first user is determined by trial and error, so that the environmental control more suitable for each individual user can be determined. Moreover, the first environmental control and the second environmental control are both environmental controls for users with attributes similar to those of the first user. Therefore, it is possible to efficiently determine the content of environmental control without accumulating a large amount of historical data for the first user.

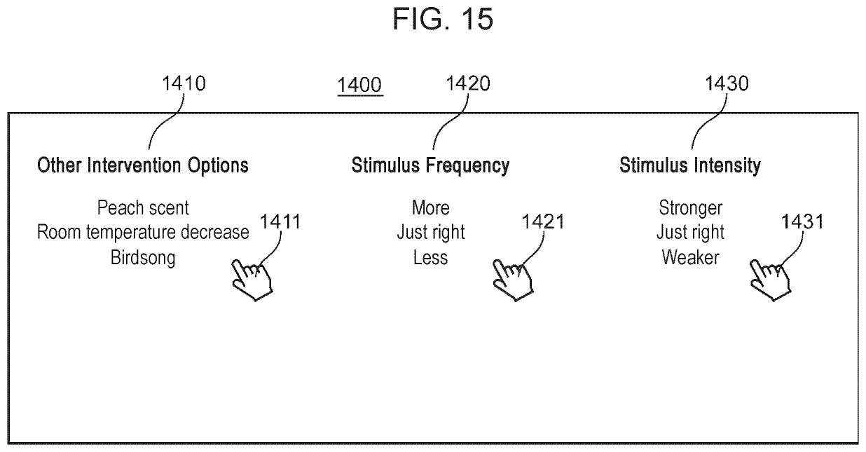

The present disclosure can adopt the following modifications.
(1) In step S22, the display output unit 11 may display a selection screen 1400 illustrated in FIG. 15 on the display of the terminal apparatus 4. When the first user continues to use the optimized intervention content, the first user may become habituated to it although the intervention effect can be achieved. Therefore, the display output unit 11 displays the selection screen 1400 on the display of the terminal apparatus 4 for selecting any one of “other intervention options”, “stimulus frequency”, and “stimulus intensity”.

The selection screen 1400 includes a selection area 1410 for “other intervention options”, a selection area 1420 for “stimulus frequency”, and a selection area 1430 for “stimulus intensity”. The selection area 1410 displays the second to fourth most effective intervention options for users with attributes similar to those of the first user. The first user selects a preferred intervention option from those displayed in the selection area 1410 by operating an icon 1411.

The selection area 1420 is an area that allows the first user to select a stimulus frequency. The details will be omitted, as they are the same as those of the selection area 802. The first user selects a preferred stimulus frequency from those displayed in the selection area 1420 by operating an icon 1421.

The selection area 1430 is an area that allows the first user to select a stimulus intensity. The selection area 1430 allows the first user to select one of “stronger”, “just right”, and “weaker”. By operating an icon 1431, the first user selects a preferred stimulus intensity from those displayed in the selection area 1430. If “stronger” is selected, the intensity of the intervention selected in the selection area 1410 is made stronger than the default value by a predetermined value. If “just right” is selected, the intensity of the intervention selected in the selection area 1410 is maintained at the default value. If “weaker” is selected, the intensity of the intervention selected in the selection area 1410 is made weaker than the default value by a predetermined value. The selected intervention, stimulus frequency, and stimulus intensity are stored in the storage 13 and determined as the intervention, stimulus frequency, and stimulus intensity for the next time the first user uses the booth 5.

The selection screen 1400 in FIG. 15 may be displayed instead of the display screens 500, 600, and 800, or may be displayed so as to be switchable with the display screens 500, 600, and 800.
(2) Although fine-tuning was performed to determine the optimal intervention in step S10, this fine-tuning may determine the normal environmental control most suitable for the first user.
(3) In step S2, the historical data of environmental controls previously performed on users with attributes similar to those of the first user was acquired from the group database 30. In this case, the environmental control determiner 17 may acquire the historical data by taking the timing into account. For example, the environmental control determiner 17 estimates the current season from the current date and time, and also estimates, from the “intervention date/time” recorded in historical data with similar attributes, the season in which the intervention was performed for each historical data. The estimated season may be a hot or cold season, or may be spring, summer, fall, or winter. The environmental control determiner 17 may acquire historical data that matches the current season from among historical data with similar attributes. In step S3, the environmental control determiner 17 may determine, as the first environmental control, the historical data with the highest intervention effect among the historical data that matches the current season.

### (Other 1)

The information processing method and the information processing system according to the present disclosure are not limited to the embodiments described above. Other embodiments obtained by combining any of the components in the embodiments described above, as well as modifications obtained by making various changes conceived by those skilled in the art to the embodiments described above, without departing from the spirit of the present disclosure, are also included in the present disclosure.

### (Other 2)

A modification of the embodiments according to the present disclosure may be as follows.

A method is executed by a computer and includes:
receiving first information indicating the amount of cerebral blood flow of a target user measured before a first device executes a process Pe1;
causing the first device to execute the process Pe1;
receiving second information indicating the amount of cerebral blood flow of the target user measured after the first device executes the process Pe1;
determining a value on the basis of the first information and the second information, the value indicating an extent to which the amount of cerebral blood flow of the target user increases after the first device executes the process Pe1, as compared to before execution of the process Pe1; and
causing the first device to execute a process Pe2 after confirming that the value is less than a predetermined value.

In the method,
a value V1 indicates an extent to which the amount of cerebral blood flow of a first user increases after execution of a first process, as compared to before the execution, . . . , and a value Vn indicates an extent to which the amount of cerebral blood flow of an n-th user increases after execution of an n-th process, as compared to before the execution;
1≤k≤n, 1≤j≤n, and k≠j are satisfied, where n is an integer greater than or equal to 2, k is an integer, and j is an integer;
a value Vk is the largest of the value V1, . . . , and the value Vn;
a value Vj is the second largest of the value V1, . . . , and the value Vn;
the process Pe1 is the same as a k-th process;
the process Pe2 is the same as a j-th process;
when the first device executes the process Pe1, a sound is output;
when the first device executes the process Pe2, an image is output; and
the first user differs from the target user, . . . , and the n-th user differs from the target user.

The present disclosure is useful in the technical field of forming a user's work environment.

What is claimed is:

1. An information processing method in a computer, the method comprising:
deriving a psychological score indicating a degree of a psychological state of a first user;
executing, on the first user, a first environmental control previously performed on users with attributes similar to attributes of the first user;
determining whether the psychological score of the first user has increased as a result of the first environmental control; and
determining, if it is determined that the psychological score has not increased, a second environmental control different from the first environmental control as the next environmental control to be executed on the first user, the second environmental control being an environmental control that was previously performed on users with attributes similar to attributes of the first user and can contribute to an increased psychological score.

2. The information processing method according to claim 1, wherein the determining the environmental control includes determining, if it is determined that the psychological score has increased, a content of the next environmental control to be executed on the first user on the basis of a content of the first environmental control.

3. The information processing method according to claim 1, wherein the first environmental control and the second environmental control each include a control that applies, to the first user, a stimulus related to at least one sense selected from the group consisting of senses of vision, hearing, smell, and touch, and include a control that changes a content of the stimulus in response to a decrease in the psychological score of the first user.

4. The information processing method according to claim 3, wherein the control that changes the content of the stimulus in response to a decrease in the psychological score of the first user includes a control that changes the content of the stimulus when the psychological score of the first user falls below a threshold determined in association with a content of the first environmental control.

5. The information processing method according to claim 4, wherein the determining the environmental control includes determining, if the psychological score has increased, an environmental control with a different threshold as a content of the next environmental control to be executed on the first user, without changing the stimulus applied by the first environmental control.

6. The information processing method according to claim 3, wherein the determining the environmental control includes determining, if it is determined that the psychological score has increased, an environmental control that applies to a user a stimulus similar to the stimulus applied by the first environmental control as a content of the next environmental control to be executed on the first user.

7. The information processing method according to claim 1, wherein the determining whether the psychological score of the first user has increased as a result of the first environmental control includes
comparing the amount of past increase in the psychological score of users with attributes similar to attributes of the first user, resulting from the first environmental control previously performed, with the amount of increase in the psychological score of the first user, resulting from the first environmental control; and
determining that the psychological score of the first user has increased as a result of the first environmental control when the amount of increase in the psychological score of the first user exceeds or is substantially the same as the amount of past increase in the psychological score.

8. The information processing method according to claim 1, wherein the second environmental control determined when it is determined that the psychological score has not increased is an environmental control that yields the second largest increase in the psychological score after the first environmental control, among environmental controls previously performed on users with attributes similar to attributes of the first user.

9. The information processing method according to claim 1, wherein the psychological score indicates a degree of user's concentration or creativity.

10. The information processing method according to claim 1, further comprising recording contents of environmental controls executed on individual users in a personal database in association with the attributes of the first user.

11. The information processing method according to claim 10, further comprising generating a group database by organizing the contents of environmental controls recorded in the personal database in accordance with the attributes of the users, the group database indicating the contents of environmental controls corresponding to the attributes.

12. The information processing method according to claim 1, further comprising presenting, to the first user, a display screen indicating the psychological score derived when the first environmental control or the second environmental control is executed.

13. An information processing system comprising a processor, wherein
the processor executes a process involving deriving a psychological score indicating a degree of a psychological state of a first user;
executing, on the first user, a first environmental control previously performed on users with attributes similar to attributes of the first user;
determining whether the psychological score of the first user has increased as a result of the first environmental control; and
determining, if it is determined that the psychological score has not increased, a second environmental control different from the first environmental control as the next environmental control to be executed on the first user, the second environmental control being an environmental control that was previously performed on users with attributes similar to attributes of the first user and can contribute to an increased psychological score.
